(19) United States
(12) Patent Application Publication
(10) Pub. No.: US 2023/0233589 A1
(43) Pub. Date: Jul. 27, 2023

(54) METHODS FOR TREATING CANCER USING A MODIFIED MONOSACCHARIDE COMPOUND

(71) Applicant: THERAONCO, MARSEILLE CEDEX 9 (FR)

(72) Inventor: Sam DUKAN, MARSEILLE (FR)

(21) Appl. No.: 18/011,771

(22) PCT Filed: Jul. 6, 2021
(86) PCT No.: PCT/EP2021/068695

§ 371 Date: Dec. 20, 2022

(30) Foreign Application Priority Data
Jul. 7, 2020 (EP) 20305771.6

(51) Int. Cl. A61K 31/7004; A61P 35/00

(52) U.S. Cl. CPC A61K 31/7004; A61P 35/00

(57) ABSTRACT

The present invention relates to the medicinal field, in particular of oncology. It relates to a modified monosaccharide compound for use in the treatment of a cancer or more specifically tumor cancers.

### FIELD OF THE INVENTION

The present invention relates to the medicinal field, in particular of oncology. It relates to a modified monosaccharide compound for use in the treatment of a cancer or more specifically tumor cancers.

### BACKGROUND OF THE INVENTION

Carbohydrates are important as signaling molecules and for cellular recognition events. They can produce multivalent interactions with carbohydrate recognition proteins (CRPs) and be used as probes of living organisms. Carbohydrates thus present many opportunities in disease diagnosis and therapy. As a consequence, the development of carbohydrate-based bioactive compounds and sensors has become an active research area. An effective and modular synthetic approach to prepare functional carbohydrates derivatives is click chemistry. In that respect, WO2016/177712 describes modified monosaccharide compounds, such as 5-azido-5-deoxy-D-arabinofuranose (also called herein “Arabinose-N3” or “Ara-N3”) in methods for labeling specifically living microorganisms.

However, modified carbohydrate/monosaccharide compounds used as probes of living organisms have never been described as potent anticancer agents.

Since cancer is one of the leading causes of death in developed countries, as cancer affects all ages, sexes, racial and ethnic groups, there is a constant need to find and develop new candidates for the treatment of cancers.

In this regard, the present inventors have found modified monosaccharide compounds that can reduce tumor growth and/or volume efficiently, treating thereby cancer and more specifically tumor cancers.

### SUMMARY OF THE INVENTION

The present invention is based on a compound of formula (I):

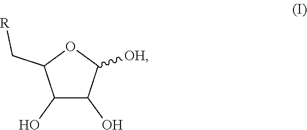

wherein R is a reactive group for click chemistry,
or a metabolite of compound of formula (I),
for use in the treatment of cancer.

More particularly, the inventors have found that a compound of formula (I) or one of its metabolite can treat cancer by slowing down tumor growth and/or reducing tumor mass (or volume).

Furthermore, it has been found that assimilation of a compound according to the invention, such as Arabinose-N3, occurs with eukaryotic cell and that such assimilation was more important in tumoral eukaryotic cells compared to non-tumoral cells (in particular for bladder, blood, skin, pancreas, brain, liver, kidney, lung, muscle, lymphocyte, prostate, stomach, breast cancer compared to non-cancer cells). Therefore, compounds of the invention present the advantage to target cancer cells more efficiently.

The present invention relates to a pharmaceutical composition comprising at least one compound of formula (I) or a metabolite thereof, in a pharmaceutically acceptable support.

In one embodiment, the present disclosure provides a method for treating cancer in a subject in need thereof comprising administering to the subject an effective amount of a compound of formula (I) or a metabolite thereof, or an effective amount of a pharmaceutical comprising the same.

### DESCRIPTION OF THE FIGURES

FIGS. 1 (a) and (b): tumor volume (mm³) with i.v. administration of 20 mg Ara-N3 (a) or oral administration of 200 mg Ara-N3 (b) in Hela tumor bearing mice following days of Hela tumor implantation (Implantation occurred at Day 0 and Ara-N3 treatment started at Day 7). Mean CTL: no treatment with Ara-N3

FIGS. 2 (a) and (b): tumor volume (mm³) with i.v. administration of 20 mg Ara-N3 (a) or oral administration of 400 mg Ara-N3 (b) in Cal33 tumor bearing mice following days of Cal33 tumor implantation (Implantation occurred at Day 0 and Ara-N3 treatment started at Day 7). Mean CTL: no treatment with Ara-N3

FIGS. 3 (a) and (b): tumor volume (mm³) with i.v. administration of 100 mg AraN3 (a) or oral administration of 200 mg Ara-N3 (b) in Panc 1 tumor bearing mice following days of Panc 1 tumor implantation (Implantation occurred at Day 0 and Ara-N3 treatment started at Day 10). Mean CTL: no treatment with Ara-N3

### DETAILED DESCRIPTION OF THE INVENTION

### Definitions

According to the present invention, the terms below have the following meanings:

As used herein, the terms “patient” and “subject” can be used interchangeably and include both humans and animals, more specifically humans.

Cancer cells are cells that divide relentlessly, forming solid tumors or flooding the blood with abnormal cells. It can thus be a solid cancer or a hematopoietic cancer, such as lymphoma or leukemia. According to a particular embodiment, the cancer is a tumor cancer.

The term “cancer” or “tumor”, as used herein, refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). Typical cancers are solid or hematopoietic cancers such as breast, brain, stomach, liver, skin, prostate, pancreatic, oesophageal, sarcoma, ovarian, endometrium, bladder, cervix uteri, rectum, colon, renal, lung or ORL cancers, pediatric tumors (neuroblastoma, glioblastoma multiforme), lymphoma, carcinoma, glioblastoma, hepatoblastoma, leukemia, myeloma, seminoma, Hodgkin or malignant hemopathies.

The reactive group R of the compound of formula (I) is a reactive group generally used in click chemistry. Click chemistry is a well-known method from a skilled person for attaching a probe or a substrate of interest to a specific biomolecule, such as a modified monosaccharide compound. Click chemistry generally implements biorthogonal reactions. The reactive group R can be defined as a reactive group involved in biorthogonal reactions. A number of chemical ligation strategies have been developed that fulfill the requirements of bioorthogonality, including the 1,3-dipolar cycloaddition between azides and cyclooctynes (also termed copper-free click chemistry), between nitrones and cyclooctynes, oxime/hydrazone formation from aldehydes and ketones, the tetrazine ligation, the isocyanide-based click reaction, and most recently, the quadricyclane ligation. As an example, an azide alkyne cycloaddition is a well-known so-called click chemistry reaction, in the presence or not of a copper catalyst, in which the azide group reacts with the alkyne group to afford a triazole. The alkyne group (—C≡C—) can be strained or not. More specifically, the alkyne group can be a terminal alkyne (i.e.: —C≡CR′, where R′ is H or a (C₁-C6) alkyl group, the alkyl group being for instance methyl, ethyl, propyl, isopropyl) or the alkyne group can be a strained alkyne, and more specifically a cyclic strained alkyne, such as cyclooctynes.

According to the invention, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated.

The present invention includes within its scope all stereoisomeric and isomeric forms of the compounds disclosed herein, including all diastereomeric isomers, racemates, enantiomers and mixtures thereof. It is also understood that the compounds described by Formula I may be present as E and Z isomers, also known as cis and trans isomers. Thus, the present disclosure should be understood to include, for example, E, Z, cis, trans, (R), (S), (L), (D), (+), and/or (−) forms of the compounds, as appropriate in each case. Where a structure has no specific stereoisomerism indicated, it should be understood that any and all possible isomers are encompassed. Compounds of the present invention embrace all conformational isomers. Compounds of the present invention may also exist in one or more tautomeric forms, including both single tautomers and mixtures of tautomers. Also included in the scope of the present invention are all polymorphs and crystal forms of the compounds disclosed herein.

The percentages are herein expressed by weight, unless otherwise specified.

### Compounds

In a particular embodiment of the invention, the compound of formula (I) includes any diastereoisomer thereof. In a further particular embodiment, the compound of formula (I) is selected in the group consisting of the following formulae:

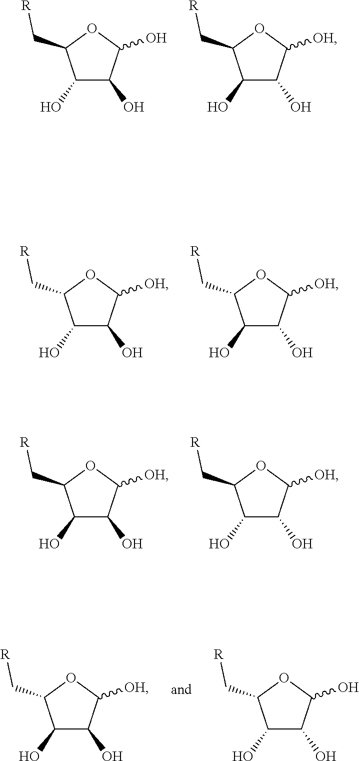

wherein R is a reactive group, such as an azide (—N₃) or an alkyne group.

According to a particular embodiment, R is a reactive group suitable for click chemistry, as detailed above. R is more specifically selected among groups consisting in or bearing the azido group (—N₃) and groups consisting in or bearing the alkyne group (—C≡C—). R is thus more specifically a group usually involved in an azide alkyne cycloaddition.

According to another embodiment, R is a strained alkyne, such as azadibenzocyclooctyne (ADIBO, DIBAC or DBCO) or tetramethoxydibenzocyclooctyne (TMDIBO). Other appropriate strained alkynes frequently used for copper-free reaction include: cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne.

Other reactive groups involved in other reactions can be cited, such as: Staudinger Ligation (first reactive group=azide and second reactive group=phosphine), copper-free click-chemistry (first reactive group=azide and second reactive group=constrained alkyne (intracyclic alkyne)), carbonyl condensation (first reactive group=aldehyde or ketone and second reactive group=hydrazide or oxyamine), thiol-ene click chemistry (first reactive group=thiol and second reactive group=alkene), nitrile-oxide-ene click chemistry (first reactive group=nitrile oxide or aldehyde, oxime, or hydroxymoyl chloride or chlororoxime and second reactive group=alkene or alkyne), nitrile imine-ene click chemistry (first reactive group=nitrile imine or aldehyde, hydrazone, or hydrazonoyl chloride or chlorohydrazone and second reactive group=alkene or alkyne), inverse electron demand Diels-Aider ligation (first reactive group=alkene and second reactive group=tetrazine), isonitrile-tetrazine click chemistry (first reactive group=isonitrile and second reactive group=tetrazine), Suzuki-Miyaura coupling (first reactive group=aryl halide and second reactive group=aryl boronate), His-tag (first reactive group=oligo-histidine and second reactive group=nickel-complex or nickel ligand). R can thus be any one of the first or second reactive groups as specified above.

According to a particular embodiment, R is an alkyne group of formula: —C≡CR′, where R′ is H or a (C₁-C6)alkyl group, the alkyl group being linear, cyclic or branched, including, but not limited to, methyl, ethyl, propyl, isopropyl. Preferably R′ is H.

According to another particular embodiment, R is a strained alkyne, and more specifically a cyclic strained alkyne, such as cyclooctynes. R can be selected in the group consisting of azadibenzocyclooctyne (ADIBO, DIBAC or DBCO) or tetramethoxydibenzocyclooctyne (TMDIBO). Other appropriate strained alkynes frequently used for copper-free reaction include: cyclooctyne (OCT), aryl-less cyclooctyne (ALO), monofluorocyclooctyne (MOFO), difluorocyclooctyne (DIFO), dibenzocyclooctyne (DIBO), dimethoxyazacyclooctyne (DIMAC), biarylazacyclooctynone (BARAC), bicyclononyne (BCN), tetramethylthiepinium (TMTI, TMTH), difluorobenzocyclooctyne (DIFBO), oxa-dibenzocyclooctyne (ODIBO), carboxymethylmonobenzocyclooctyne (COMBO), or benzocyclononyne.

According to another embodiment, R is selected among groups consisting in or bearing the azido group (—N₃). More specifically, R is the azido group.

According to a particular embodiment, the compound of formula (I) is selected in the group consisting of the following formulae:

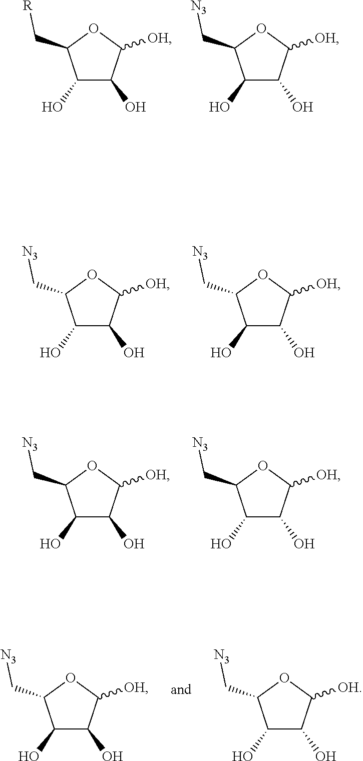

In a preferred embodiment, the compound of formula (I) is 5-azido-5-deoxy-D-arabinofuranose (arabinose-N₃ or also named Ara-N₃), in particular having the following formula (II):

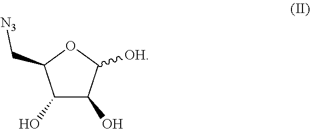 [SMILES: [N-]=[N+]=NC[C@H]1OC(O)[C@@H](O)[C@@H]1O]

In another specific embodiment, the compound of formula (I) has the following formula (III):

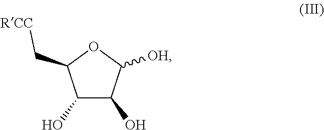

wherein CCR′ is an alkyne group as defined above, and more specifically R′ is H or a (C₁-C6)alkyl group, the alkyl group being linear, cyclic or branched, including, but not limited to, methyl, ethyl, propyl, isopropyl. Preferably R′ is H.

In a further particular embodiment of the invention, the compound for use according to the invention is a metabolite of a compound of formula (I), more specifically a metabolite of a compound of formula (II), or (III). In a preferred embodiment, the metabolite is a metabolite of arabinose, preferably L-arabinose.

As used herein a “metabolite of a compound of formula (I)” is a compound from the pentose phosphate pathway, wherein said compound further comprises a reactive group R, as defined above, and more specifically R is selected among groups consisting in or bearing the azido group (—N₃) and groups consisting in or bearing the alkyne group (—C≡C—). The pentose phosphate pathway is described in many reviews, such as in Mujaji B. W., “the pentose phosphate pathway revised” in Biochemical education, 8(3) 1980, pp 76-78, or Jin and Zhou: Pentose Phosphate Pathway In Cancer—ONCOLOGY LETTERS 17: 4213-4221 (2019 DOI: 10.3892/ol.2019.10112). In a particular embodiment, a metabolite of a compound of formula (I) includes ribose, ribose 5-P, ribulose, ribulose 5-P, L-ribulose, L-ribulose 5-P, arabinitol, L-arabinitol, lyxose (L or D-lyxose), xylulose, xylulose-5-P, D-xylulose, D-xylulose-5-P, L-xylulose, D-xylulose, or xylitol, said compound further comprising an azide (N₃) or an alkyne group, as defined above. In a preferred embodiment, a metabolite of a compound of formula (I) is a metabolite of arabinose, further comprising an azide (N₃) or an alkyne group, as defined above. In a preferred embodiment, a metabolite of arabinose includes L-ribulose, L-ribulose 5-P, or D-xylulose-5-P, said compound further comprising an azide (N₃) or an alkyne group, as defined above. In a further preferred embodiment, a “metabolite of compound of formula (I)” includes L-arabinitol, L-xylulose, xylitol, D-xylulose, or D-xylulose-5-P, said compound further comprising an azide (N₃) or an alkyne group, as defined above.

According to a specific embodiment, the compound of formula (I) to be used as an active ingredient to treat cancer is not attached on the surface of eukaryotic cells or not bond to any compound, such as a compound having a chemical group able to react with the azide group (—N₃) or the alkyne group of the compound of formula (I) via a click chemistry reaction.

### Pharmaceutical Composition

The present invention relates to a pharmaceutical composition comprising at least one compound of formula (I) or a metabolite thereof as above defined, in a pharmaceutically acceptable support. According to the present invention, the compound of formula (I) is a therapeutically active ingredient, or preferably the sole active ingredient.

In a particular aspect, the compound of formula (I) or a metabolite thereof as defined above, as an active ingredient, more specifically for use to treat cancer, is not attached on the surface of eukaryotic cells or not bond to any compound, such as a compound having a chemical group able to react with the azide group (—N₃) of the compound of formula (I) via a click chemistry reaction.

The pharmaceutical compositions contemplated herein include a pharmaceutically acceptable carrier in addition to the anti-cancer drug, which at least one compound of formula (I). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. The pharmaceutical compositions can be administered enterally or parenterally. The pharmaceutical composition can be orally, intravenously, intraperitoneally, intratracheally, intratumorally, intramuscularly, endoscopically, intralesionally, percutaneously, subcutaneously, or regionally administered. Administration includes direct injection or perfusion. For example, for parenteral administration, the active compounds(s) may be formulated in a unit dosage form for injection in vehicles such as saline, dextrose solution, serum albumin and Ringer's solution. The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations suitable for parenteral administration conveniently comprise a sterile oily or aqueous preparation of the active ingredient which is preferably isotonic with the blood of the recipient.

The carrier must be “acceptable” in the sense of being compatible with the other ingredients of the formulations and not deleterious to the recipient thereof. The pharmaceutical compositions are advantageously applied by injection or intravenous infusion of suitable sterile solutions. Methods for the safe and effective administration of most of these anticancer drugs are known to those skilled in the art. In addition, their administration is described in the standard literature.

### Treatment

The cancer treated according to the invention a solid or hematopoietic cancer, more specifically a solid cancer. According to a particular embodiment, the treated cancer is chosen among rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer. More specifically, the cancer is selected from the group consisting of bladder, blood, skin, cervical, pancreas, brain, liver, kidney, lung, muscle, lymphocyte, prostate, stomach, and breast cancer. According to a more particular embodiment, the cancer is selected from the group consisting of: bladder, blood, colon, cervical, stomach, breast, lung, skin, head and neck and pancreas cancers. In a particular embodiment, the treated cancer of the invention is selected in the group consisting of head and neck cancer, such as tongue squamous cell carcinoma, pancreas and cervical cancer.

The compound of formula (I) or a metabolite of compound of formula (I) or the pharmaceutical composition of the invention can be used in cancer therapy.

A preferred embodiment of the invention is a compound of formula (I) or a metabolite thereof as defined herein, or a pharmaceutical composition as defined herein, for use in the treatment of a cancer as above defined.

More specifically, the compound of formula (I) or a metabolite thereof as defined herein is for use in the treatment of cancer preferably by slowing down tumor growth and/or reducing tumor mass (or volume), or by reducing the doubling time of tumor cells.

A further preferred embodiment, is a method for treating a cancer in a subject in need thereof, comprising administering a therapeutically effective amount of a compound of formula (I) or a metabolite thereof as defined herein or a therapeutically effective amount of a pharmaceutical composition comprising the same.

A further preferred embodiment, is a use of a compound of formula (I) or a metabolite thereof as defined herein, or a pharmaceutical composition as defined herein, for the manufacture of a medicament for the treatment of cancer.

The term “treating” or “treatment” refers herein to executing a protocol, which may include administering one or more drugs to a patient, in an effort to alleviate signs or symptoms of the cancer, which can be at any stage. Desirable effects of treatment include decreasing the rate of cancer progression, ameliorating or palliating the cancer state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The term “effective”, as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

The amount to be administered is not subject to definite bounds, but it will usually be an effective amount, or the equivalent on a molar basis of the pharmacologically active free form produced from a dosage formulation upon the metabolic release of the active drug to achieve its desired pharmacological and physiological effects. An oncologist skilled in the art of cancer treatment will be able to ascertain appropriate protocols for the effective administration of the compounds of the present invention, such as by referring to the earlier published studies on compounds found to have anti-tumor properties.

Further aspects and advantages of the present invention will be described in the following examples, which should be regarded as illustrative and not limiting.

### EXAMPLES

### Example 1: Synthesis of Compounds

### Materials and Methods:

Thin layer chromatography was performed over Merck 60 F254 with detection by UV, and/or by charring with sulphuric acid or KMnO₄ or phosphomolybdic acid solutions. Silica gel 60 40-63 Mm was used for flash column chromatography.

NMR spectra were taken on Bruker Avance 300 or 500 MHz spectrometers, using the residual protonated solvent as internal standard. Chemical shifts δ are given in parts per million (ppm) and coupling constants are reported as Hertz (Hz). Splitting patterns are designated as singlet (s), doublet (d), triplet (t), doublet of doublet (dd), doublet of doublet of doublet (ddd). Splitting patterns that could not be interpreted or easily visualized are designated as multiplet (m).

Mass spectra were taken on a Waters LCT Premier XE (ToF), with electrospray ionization in the positive (ESI+) or in the negative (ESI−) mode of detection.

IR-FT spectra were recorded on a Perkin Elmer Spectrum 100 spectrometer. Characteristic absorptions are reported in cm⁻¹.

Specific optical rotations were measured at 20° C. with an Anton Paar MCP 300 polarimeter in a 10-cm cell at 20° C. and 589 nm.

All biological and chemical reagents were of analytical or cell culture grade, obtained from commercial sources, and used without further purifications.

Ara-N₃ was synthesized according to the following procedure:

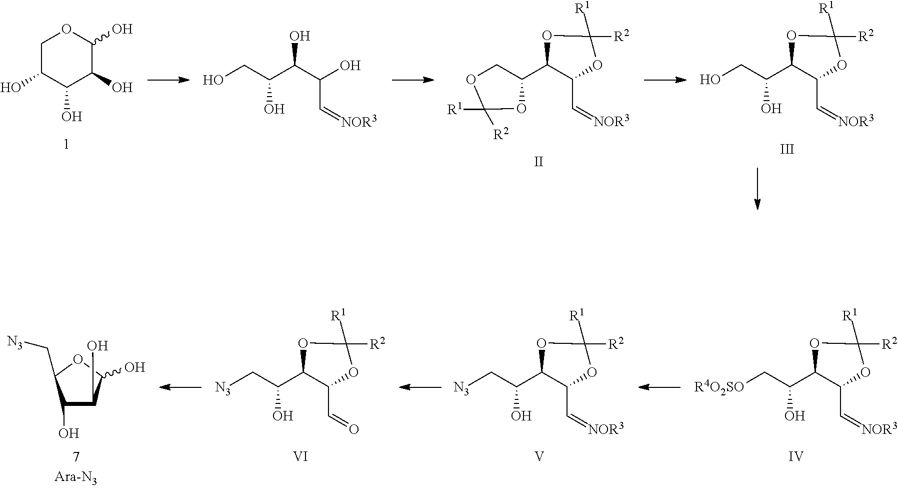

with R¹, R², and R³ being a methyl group.

### 2:3,4:5-diisopropylidene-D-arabinose O-methyloxime (Compound II)

To a solution of D-(−)-arabinose (4.00 g, 26.6 mmol, 1.0 eq.) in dry pyridine (90 mL) was added methoxyamine hydrochloride (2.72 g, 32.0 mmol, 1.2 eq.) and the mixture was stirred at room temperature for 15 hours. Solvents were removed under reduced pressure and the residue was co-evaporated with toluene three times. The residue was resuspended in 2,2-dimethoxypropane (100 mL) and 7-toluenesulfonic acid (1.01 g, 5.33 mmol, 0.2 eq.) was added and the suspension was heated to reflux for 4 hours followed by further 15 hours of stirring at room temperature. The reaction mixture was filtered over Celite® and solvents were evaporated. The residue was dissolved in ethyl acetate (200 mL) and was washed with saturated aq. NaCl solution (2×150 mL). Purification by silica flash column chromatography (cyclohexane/ethyl acetate 9:1) yields a mixture of isomers 2:3,4:5-diisopropylidene-D-arabinose O-methyloxime (IIE/IIZ) and an unknown impurity (NMR ratio 6:1:0.4, 5.83 g) as colorless oil. This mixture was used without further purification in the next step. An aliquot of pure (2E) was obtained by a second flash column chromatography (dichloromethane/MTBE 98:2) and characterized.

Isomer (IIE):

Rf (CH₂Cl₂/MTBE 98:2): 0.35.

IR (cm⁻¹): 2987, 2939, 2900, 2821, 1631, 1456, 1381, 1371, 1241, 1212, 1150, 1065, 1038, 887, 842.

¹H-NMR (500 MHz, CDCl₃) δ: 7.35 (d, 1H, J_{1,2} 6.3 Hz, H-1); 4.46 (dd, 1H, J_{2,3} 7.1, J_{1,2} 6.3 Hz, H-2); 4.13 (ddd, 1H, J_{3,4} 6.9, J_{4,5a} 6.1, J_{4,5b} 4.8 Hz, H-4); 4.08 (dd, 1H, J_{5a,5b} 8.5, J_{4,5a} 6.1 Hz, H-5a); 3.97 (d, 1H, J_{2,3} 7.1, J_{3,4} 6.9 Hz, H-3); 3.94 (dd, 1H, J_{5a,5b} 8.5, J_{4,5b} 4.8 Hz, H-5b); 3.85 (s, 3H, CH₃—O); 1.40 (s, 3H, CH₃—C); 1.38 (s, 6H, 2 CH₃—C); 1.32 (s, 3H, CH₃—C).

¹³C-NMR (125 MHz, CDCl₃) δ: 147.8 (C-1); 110.8 (C-6); 110.0 (C-7); 79.4 (C-3); 76.7 (C-2); 76.6 (C-4); 67.1 (C-5); 62.1 (CH₃—O); 27.1, 27.0, 26.9, 25.4 (4 CH₃—C). HRMS (ESI⁺): [M+H]⁺ (C₁₂H₂₂NO₅⁺) Calc. m/z: 260.1492, found: 260.1502.

### 2:3-isopropylidene-D-arabinose O-methyloxime (Compound III)

A solution of 2:3,4:5-diisopropylidene-D-arabinose O-methyloxime (II) (IIE/IIZ) and impurity (1.50 g) in 80% (v/v) aqueous acetic acid (30 mL) was heated to 40° C. at 200 mbar of pressure on a rotavap. After 2.5 hours solvents were removed under reduced pressure and the residue was co-evaporated with toluene. A mixture of isomers 2:3-isopropylidene-D-arabinose O-methyloxime (IIIE/IIIZ) (NMR ratio 4:1, 876 mg, 58% over 3 steps) were obtained after silica gel flash column chromatography (cyclohexane/ethyl acetate 1:1) as colorless oil. Purity of more than 95% by NMR.

Rf (cyclohexane/ethyl acetate 1:1): 0.24.

IR (cm⁻¹): 3409, 2939, 1373, 1216, 1040, 885.

HRMS (ESI⁺): [M+H]⁺ (C₉H₁₈NO₅⁺) Calc. m/z: 220.1179, found: 220.1184.

Isomer (IIIE):

¹H-NMR (500 MHz, CDCl₃) δ: 7.44 (d, 1H, J_{1,2} 5.5 Hz, H-1); 4.56 (dd, 1H, J_{2,3} 7.4, J_{1,2} 5.5 Hz, H-2); 4.07 (dd, 1H, J_{2,3} 7.4, J_{3,4} 5.6 Hz, H-3); 4.13 (ddd, 1H, J_{3,4} 5.6, J_{4,5} 5.1, J_{4,5} 4.7 Hz, H-4); 3.84 (s, 3H, CH₃—O); 3.72-3.68 (m, 2H, 2H-5); 1.42 (s, 3H, CH₃—C); 1.38 (s, 3H, CH₃—C).

¹³C-NMR (125 MHz, CDCl₃) δ: 149.1 (C-1); 110.3 (C-6); 79.4 (C-3); 75.0 (C-2); 71.6 (C-4); 63.4 (C-5); 62.2 (CH₃—O); 26.9, 26.7 (2 CH₃—C).

Isomer (IIIZ):

¹H-NMR (500 MHz, CDCl₃) δ: 6.86 (d, 1H, J_{1,2} 5.9 Hz, H-1); 4.95 (dd, 1H, J_{2,3} 7.7, J_{1,2} 5.9 Hz, H-2); 3.92 (s, 3H, CH₃—O); 3.87 (dd, 1H, J_{2,3} 7.7, J_{3,4} 6.9 Hz, H-3); 3.82-3.75 (m, 2H, H-4, H-5a); 3.72-3.68 (m, 1H, H-5b); 1.40 (2s, 6H, 2 CH₃—C).

¹³C-NMR (125 MHz, CDCl₃) δ: 151.0 (C-1); 110.9 (C-6); 80.4 (C-3); 72.9 (C-2); 72.7 (C-4); 63.5 (C-5); 62.8 (CH₃—O); 27.0, 26.5 (2 CH₃—C).

### 2:3-isopropylidene-5-O-methanesulfonyl-D-arabinose O-methyloxime (Compound IV)

To a solution of 2:3-isopropylidene-D-arabinose O-methyloxime (III) (IIIE/IIIZ) (100 mg, 0.46 mmol, 1.0 eq.) in dry pyridine (2.0 mL) at −20° C., mesyl chloride (0.10 mL, 1.37 mmol, 3.0 eq.) was added and the reaction mixture was stirred for 1.5 hours at −20° C. After quenching the reaction with CH₃OH (0.3 mL), solvents were removed under vacuum. The resulting residue was purified by silica flash column chromatography (cyclohexane/ethyl acetate 6:4) to yield a mixture of 2:3-isopropylidene-5-O-methanesulfonyl-D-arabinose O-methyloxime (IVE/IVZ) (NMR ratio 4:1, 110 mg, 81%) as colorless oil. An aliquot of pure (IVE) isomer was obtained by flash column chromatography (dichloromethane/diethyl ether 9:1) and characterized. Purity of more than 95% by NMR.

Rf (cyclohexane/ethyl acetate 6:4): 0.24.

IR (cm⁻¹): 3500, 2989, 2941, 2824, 1631, 1458, 1350, 1215, 1170, 1067, 1033, 959, 887, 863, 833.

HRMS (ESI⁺): [M+H]⁺ (C₁₀H₂₀NO₇S⁺) Calc. m/z: 298.0955, found: 298.0947.

Isomer (IVE):

Rf (cyclohexane/ethyl acetate 6:4): 0.20.

¹H-NMR (500 MHz, CDCl₃) δ: 7.42 (d, 1H, J_{1,2} 5.6 Hz, H-1); 4.56 (dd, 1H, J_{2,3} 6.8, J_{1,2} 5.6 Hz, H-2); 4.41 (dd, 1H, J_{5a,5b} 11.0, J_{4,5a} 2.7 Hz, H-5a); 4.28 (dd, 1H, J_{5a,5b} 11.0, J_{4,5b} 5.7 Hz, H-5b); 4.03 (ddd, 1H, J_{3,4} 7.0, J_{4,5b} 5.7, J_{4,5a} 2.7 Hz, H-4); 4.01 (dd, 1H, J_{2,3} 6.8, J_{3,4} 7.0 Hz, H-3); 3.85 (s, 3H, CH₃—O); 3.06 (s, 3H, CH₃—S); 1.41 (s, 3H, CH₃—C); 1.39 (s, 3H, CH₃—C).

¹³C-NMR (125 MHz, CDCl₃) δ: 148.2 (C-1); 110.9 (C-6); 77.9 (C-3); 76.2 (C-2); 70.9 (C-5); 70.8 (C-4); 62.3 (CH₃—O); 37.8 (CH₃—S); 27.0, 26.9 (2 CH₃—C).

Isomer (IVZ):

¹H-NMR (300 MHz, CDCl₃) δ: 6.87 (d, 1H, J_{1,2} 5.9 Hz, H-1); 4.96 (dd, 1H, J_{2,3} 7.3, J_{1,2} 5.9 Hz, H-2); 4.45 (dd, 1H, J_{5a,5b} 11.4, J_{4,5a} 2.4 Hz, H-5a); 4.29 (dd, 1H, J_{5a,5b} 11.4, J_{4,5b} 7.9 Hz, H-5b); 3.98 (ddd, 1H, J_{4,5b} 7.9, J_{4,3} 7.5, J_{4,5a} 2.4 Hz, H-4); 3.93 (s, 3H, CH₃—O); 3.84 (dd, 1H, J_{3,4} 7.5, J_{3,2} 7.3 Hz, H-3); 3.06 (s, 3H, CH₃—S); 1.40 (s, 3H, CH₃—C); 1.39 (s, 3H, CH₃—C).

¹³C-NMR (75 MHz, CDCl₃) δ: 150.7 (C-1); 111.2 (C-6); 79.1 (C-3); 72.9 (C-2); 71.3 (C-5); 70.9 (C-4); 62.9 (CH₃—O); 37.9 (CH₃—S); 27.0, 26.6 (2 CH₃—C).

### 5-azido-5-deoxy-2:3-isopropylidene-D-arabinose O-methyloxime (Compound V)

To a solution of (IVE/IVZ) (810 mg, 2.72 mmol, 1.0 eq.) in N,N-dimethylformamide (30.0 mL, 0.10 M), sodium azide (531 mg, 8.17 mmol, 3.0 eq.) was added and the reaction mixture was heated at 80° C. for 15 hours. Solvent was then removed under reduced pressure and the residue was purified by flash column chromatography (cyclohexane/ethyl acetate 9:1) to yield a mixture of 5-azido-5-deoxy-2:3-isopropylidene-D-arabinose O-methyloxime (VE/VZ) (NMR ratio 7:3, 637 mg, 96%) as yellowish oil. A fraction of (VE) was isolated by flash column chromatography (dichloromethane/MTBE 97:3) for its characterizations. Purity of more than 95% by NMR.

Rf (cyclohexane/ethyl acetate 8:2): 0.30.

IR (cm⁻¹): 3458, 2989, 2939, 2823, 2100, 1630, 1443, 1373, 1213, 1164, 1066, 1036, 885, 865.

HRMS (ESI⁺): [M+H]⁺ (C₉H₁₇N₄O₄⁺) Calc. m/z: 245.1245, found: 245.1250.

Isomer (VE):

Rf (dichloromethane/MTBE 97:3): 0.23.

¹H-NMR (500 MHz, CDCl₃) δ: 7.42 (d, 1H, J_{1,2} 5.8 Hz, H-1); 4.54 (dd, 1H, J_{2,3} 7.2, J_{1,2} 5.8 Hz, H-2); 3.98 (dd, 1H, J_{2,3} 7.2, J_{3,4} 6.4 Hz, H-3); 3.92 (dddd, 1H, J_{3,4} 6.4, J_{4,5b} 6.2, J_{4,OH} 3.9, J_{4,5a} 3.8 Hz, H-4); 3.85 (s, 3H, CH₃—O); 3.46 (dd, 1H, J_{5a,5b} 12.5, J_{4,5a} 3.8 Hz, H-5a); 3.42 (dd, 1H, J_{5a,5b} 12.5, J_{4,5b} 6.2 Hz, H-5b); 2.61 (d, 1H, J_{4,OH} 3.9 Hz, OH); 1.41 (s, 3H, CH₃—C); 1.39 (s, 3H, CH₃—C).

¹³C-NMR (125 MHz, CDCl₃) δ: 148.4 (C-1); 110.6 (C-6); 78.8 (C-3); 75.8 (C-2); 71.5 (C-4); 62.3 (CH₃—O); 53.7 (C-5); 27.0, 26.8 (2 CH₃—C).

Isomer (VZ):

¹H-NMR (500 MHz, CDCl₃) δ: 6.86 (d, 1H, J_{1,2} 6.1 Hz, H-1); 4.94 (dd, 1H, J_{2,3} 7.2, J_{1,2} 6.1 Hz, H-2); 3.93 (s, 3H, CH₃—O); 3.87 (ddd, 1H, J_{3,4} 7.5, J_{4,5b} 6.4, J_{4,5a} 2.8 Hz, H-4); 3.82 (dd, 1H, J_{3,4} 7.5, J_{2,3} 7.2 Hz, H-3); 3.47 (dd, 1H, J_{5a,5b} 12.8, J_{4,5a} 2.8 Hz, H-5a); 3.39 (dd, 1H, J_{5a,5b} 12.8, J_{4,5b} 6.4 Hz, H-5b); 1.40 (s, 3H, CH₃—C); 1.38 (s, 3H, CH₃—C).

¹³C-NMR (75 MHz, CDCl₃) δ: 150.8 (C-1); 111.0 (C-6); 80.0 (C-3); 72.9 (C-2); 72.5 (C-4); 62.8 (CH₃—O); 53.5 (C-5); 26.9, 26.5 (2 CH₃—C).

### 5-azido-5-deoxy-2:3-isopropylidene-D-arabinose (Compound VI)

To a solution of (VE/VZ) (820 mg, 3.36 mmol, 1.0 eq.) in 80% (v/v) aqueous acetic acid (120 mL), formaldehyde (0.8 mL) was added and the reaction mixture was stirred for 1 hour at room temperature. Solvents were removed under reduced pressure and co-evaporation with toluene was done to assure complete elimination of acetic acid. The crude compound 5-azido-5-deoxy-2:3-isopropylidene-D-arabinose (VI) (682 mg) was obtained.

Colorless oil

Rf (cyclohexane/ethyl acetate 7:3): 0.56.

IR (cm⁻¹): 3408, 2988, 2936, 2100, 1733, 1440, 1373, 1238, 1213, 1164, 1063, 863.

¹H-NMR (500 MHz, CDCl₃) δ: 9.79 (d, 1H, J_{1,2} 1.2 Hz, H-1); 4.41 (dd, 1H, J_{2,3} 6.4, J_{1,2} 1.2 Hz, H-2); 4.04 (dd, 1H, J_{2,3} 6.4, J_{3,4} 6.1 Hz, H-3); 3.90 (ddd, 1H, J_{4,5b} 6.4, J_{3,4} 6.1, J_{4,5a} 3.4 Hz, H-4); 3.51 (dd, 1H, J_{5a,5b} 12.8, J_{4,5a} 3.4 Hz, H-5a); 3.43 (dd, 1H, J_{5a,5b} 12.8, J_{4,5b} 6.4 Hz, H-5b); 1.47 (s, 3H, CH₃—C); 1.37 (s, 3H, CH₃—C).

HRMS (ESI⁺): [2M+Na]⁺ (C₁₆H₂₆N₆NaO₈⁺) Calc. m/z: 453.1704, found: 453.1726.

### 5-azido-5-deoxy-D-arabinofuranose (Compound VII, Ara-N₃)

To a solution of 5-azido-5-deoxy-2:3-isopropylidene-D-arabinose (VI) (100 mg) in a mixture of CH₂Cl₂/H₂O (20:1, 21 mL), trifluoroacetic acid (1 mL) was added and the mixture was stirred at room temperature for 1 hour. Solvents were then evaporated, the crude residue was resuspended in water and lyophilized. After silica flash column chromatography (dichloromethane/methanol 92:8), the compound 5-azido-5-deoxy-D-arabinofuranose or Ara-N₃ (VII) (50 mg, 58% over 2 steps from compound (V)) was obtained as a mixture of/β anomers (NMR ratio 55:45) as a colorless oil. Purity of more than 95% by NMR.

Rf (dichloromethane/methanol 92:8): 0.28.

IR (cm⁻¹): 3367, 2106, 1281, 1040.

HRMS (ESI⁺): [M+H-N₂]⁺ (C₅H₁₀NO₄⁺) Calc. m/z: 148.0604, found: 148.0610.

Anomer Alpha (VIIα):

¹H-NMR (500 MHz, D20) δ: 5.24 (d, 1H, J_{1,2} 2.9 Hz, H-1); 4.17 (ddd, 1H, J_{3,4} 6.4, J_{4,5b} 5.8, J_{4,5a} 3.5 Hz, H-4); 4.01 (dd, 1H, J_{2,3} 4.6, J_{1,2} 2.9 Hz, H-2); 3.97 (dd, 1H, J_{3,4} 6.4, J_{3,2} 4.6 Hz, H-3); 3.64 (dd, 1H, J_{5a,5b} 13.6, J_{4,5a} 3.5 Hz, H-5a); 3.44 (dd, 1H, J_{5a,5b} 13.6, J_{4,5b} 5.8 Hz, H-5b).

¹³C-NMR (125 MHz, D20) δ: 101.0 (C-1); 81.3 (C-4); 81.2 (C-2); 76.3 (C-3); 51.5 (C-5).

Anomer Beta (VIIβ):

¹H-NMR (500 MHz, D20) δ: 5.28 (br d, 1H, J_{1,2} 3.1 Hz, H-1); 4.10-4.05 (m, 2H, H-2, H-3); 3.89 (ddd, 1H, J_{3,4} 7.1, J_{4,5b} 6.5, J_{4,5a} 3.5 Hz, H-4); 3.59 (dd, 1H, J_{5a,5b} 13.3, J_{4,5a} 3.5 Hz, H-5a); 3.42 (dd, 1H, J_{5a,5b} 13.3, J_{4,5b} 6.5 Hz, H-5b).

¹³C-NMR (125 MHz, D20) δ: 95.2 (C-1); 79.6 (C-4); 75.8 (C-2); 74.7 (C-3); 52.6 (C-5).

### Example 2: In Vivo Treatment with Ara-N₃ in Mice

### Material and Methods:

### Animal Model

45 female NMRI nude mice, 6 weeks old.

Day 0:
8 million Panc-1 cells in 200 μL of PBS implanted subcutaneously in the right flank (n=15 mice)
10 million Hela cells in 200 μL of PBS implanted subcutaneously in the right flank (n=15 mice)
5 million Cal33 cells in 200 μL of PBS implanted subcutaneously in the right flank (n=15 mice)

Clinical follow-up: animals weight and tumor volume: 2-3 times a week.

The assays were carried out in accordance with the application for authorization of animal experiments submitted to the Ministry of Research and the Ethics Committee

Ara-N3 Treatment

For each cell line, 3 mice were not treated by Ara-N₃, as control (CTL)

Intravenous Injections (IV)

3 doses of Ara-N₃ were assessed:

Hela and Cal33 Cell Lines
cumulative dose of 20 mg (5 mg/mouse/inj repeated 1 day/2 for 8 days (4 times)) Panc-1 cell line
cumulative dose of 100 mg (9 mg/mouse/injection repeated 1 day/2 for 21 days (11 times))

Drinking Water

Hela Cell Lines
cumulative dose of 200 mg per mouse: 25 mg/day (8 mL/day @ 3.13 mg/mL) over 8 days

Cal33 Cell Lines
cumulative dose of 400 mg per mouse: 50 mg/day (8 mL/day @ 6.25 mg/mL) over 8 days

Panc-1 Cell Line
cumulative dose of 200 mg per mouse: 9.5 mg/day (8 mL/day @ 1.19 mg/mL) over 21 days

### Results:

### Clinical Follow Up

The introduction of Ara-N₃ in drinking water (up to 6.25 mg/mL) did not alter the water consumption of the mice.
Treatment with Ara-N₃, either in drinking water or by intravenous injections, did not induce any body weight loss.

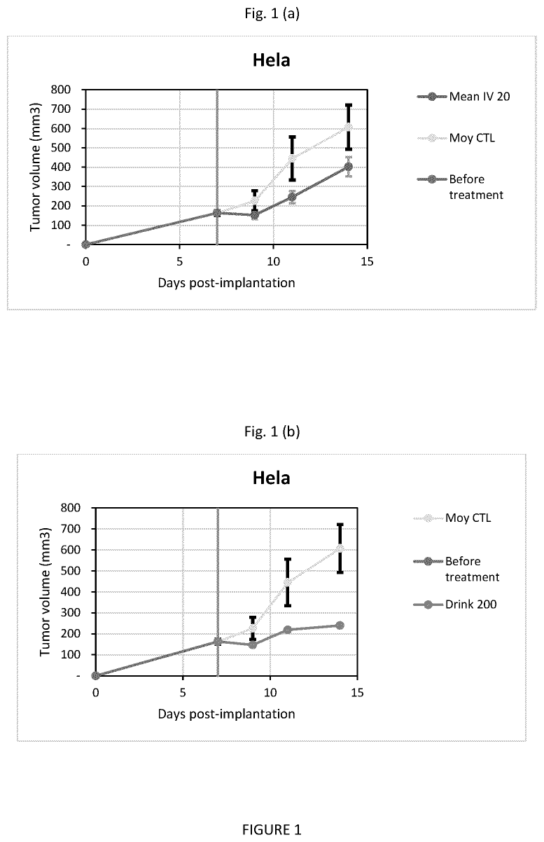

Results of tumor volume with i.v. administration of 20 mg Ara-N₃ or oral administration of 200 mg Ara-N₃ in Hela tumor bearing mice are presented on FIGS. 1 (a) and (b).

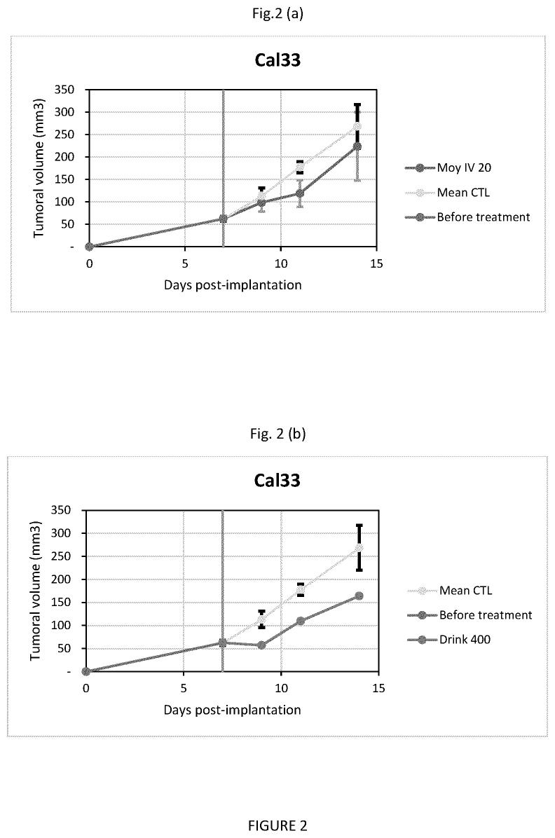

Results of tumor volume with i.v. administration of 20 mg Ara-N₃ or oral administration of 400 mg Ara-N₃ in Cal33 tumor bearing mice are presented on FIGS. 2 (a) and (b).

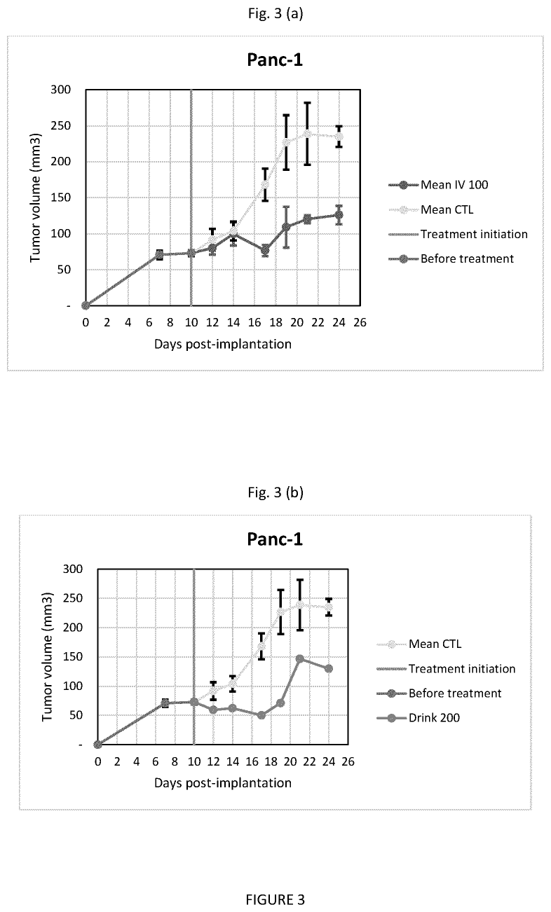

Results of tumor volume with i.v. administration of 100 mg Ara-N₃ or oral administration of 200 mg Ara-N₃ in Panc 1 tumor bearing mice are presented on FIGS. 3 (a) and (b).

### CONCLUSIONS

Treatment with Ara-N₃ by intravenous injections (i.v.) induced slower tumor growth for Hela, PANC-1 and Cal33 lines.
Ara-N₃ oral (drinking water) treatments also reduced tumor volumes compared to the non-treated control group.
Ara-N₃ i.v. treatment increased the doubling times of the Hela, PANC-1 and Cal33 lines when compared to the non-treated control group.

1.-13. (canceled)

14. A method for treating cancer in a subject in need thereof, comprising administering to the subject an effective amount of a compound of formula (I): 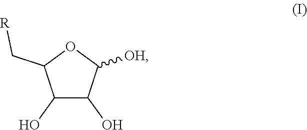
wherein R is a reactive group for click chemistry,
or an effective amount of a metabolite of the compound of formula (I).

15. The method of claim 14, wherein R is selected from the group consisting of azido group (—N₃), groups bearing an azido group (—N₃), alkyne group (—C≡C—), and groups bearing an alkyne group (—C≡C—).

16. The method of claim 14, wherein R is the azido group (—N₃).

17. The method of claim 14, wherein the compound of formula (I) is selected from the group consisting of the following formulae: 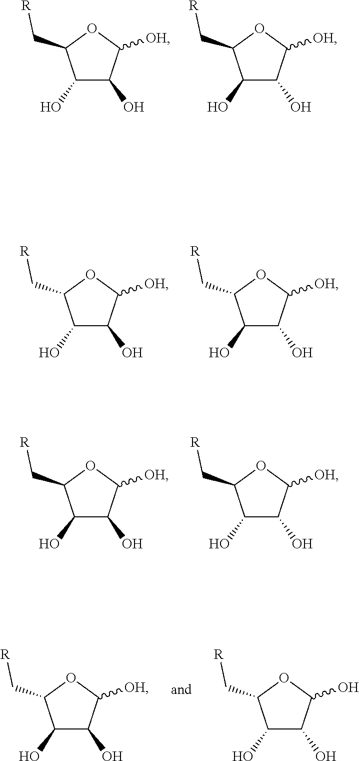
wherein R is as defined in claim 14.

18. The method of claim 14, wherein the compound of formula (I) is 5-azido-5-deoxy-D-arabinofuranose having the following formula:

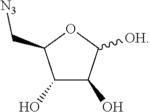 [SMILES: [N-]=[N+]=NC[C@H]1OC(O)[C@@H](O)[C@@H]1O]

19. The method of claim 14, wherein the metabolite of the compound of formula (I) is selected from the group consisting of ribose, ribose 5-P, ribulose, ribulose 5-P, L-ribulose, L-ribulose 5-P, arabinitol, L-arabinitol, xylulose, xylulose-5-P, D-xylulose, D-xylulose-5-P, L-xylulose, D-xylulose, and xylitol, said metabolite further comprising an azide (N₃) or an alkyne group.

20. The method of claim 14, wherein the cancer is selected from the group consisting of rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer.

21. A pharmaceutical composition comprising at least one modified monosaccharide compound of formula (I), or a metabolite of the compound of formula (I), in a pharmaceutically acceptable support, wherein the formula (I) is as follows: 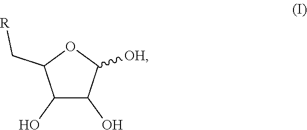
wherein R is a reactive group for click chemistry.

22. The pharmaceutical composition of claim 21, wherein R is selected from the group consisting of azido group (—N₃), groups bearing an azido group (—N₃), alkyne group (—C≡C—), and groups bearing an alkyne group (—C≡C—).

23. The pharmaceutical composition of claim 21, wherein the compound of formula (I) is selected from the group consisting of the following formulae: 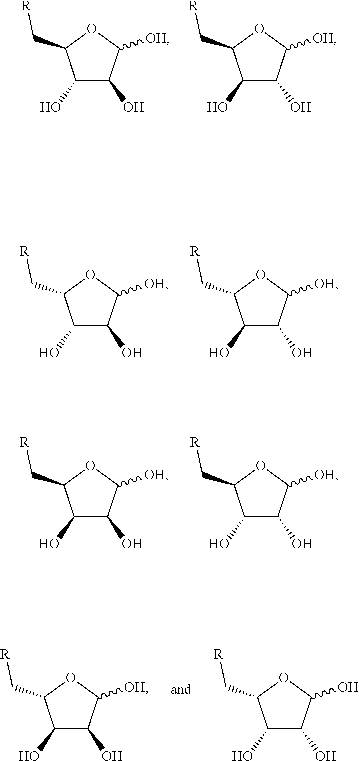
wherein R is an azide (N₃) or an alkyne group.

24. The pharmaceutical composition of claim 23, wherein R is an azide.

25. The pharmaceutical composition of claim 21, wherein the compound of formula (I) is 5-azido-5-deoxy-D-arabinofuranose having the following formula:

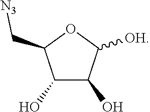 [SMILES: [N-]=[N+]=NC[C@H]1OC(O)[C@@H](O)[C@@H]1O]

26. A method for treating cancer in a subject in need thereof, comprising administering to the subject an effective amount of the pharmaceutical composition of claim 21.

27. The method of claim 26, wherein the cancer is selected from the group consisting of rectal cancer, colorectal cancer, stomach cancer, head and neck cancer, thyroid cancer, cervical cancer, uterine cancer, breast cancer, ovarian cancer, brain cancer, lung cancer, skin cancer, bladder cancer, blood cancer, renal cancer, liver cancer, prostate cancer, multiple myeloma, and endometrial cancer.
